(19) United States
(12) Patent Application Publication
(10) Pub. No.: US 2025/0066282 A1
(43) Pub. Date: Feb. 27, 2025

(54) NOVEL ANALOGS OF VALPROIC ACID AND METHODS OF MEDICAL TREATMENT USING THE SAME

(71) Applicant: Cereno Scientific AB, Moelndal (SE)

(72) Inventors: Tomas Fex, Vaestra Froelunda (SE); Jonas Faijerson Saljoe, Goteborg (SE)

(21) Appl. No.: 18/721,594

(22) PCT Filed: Dec. 20, 2022
(86) PCT No.: PCT/GB2022/053318

§ 371 Date: Jun. 18, 2024

(30) Foreign Application Priority Data
Dec. 20, 2021 (GB) 2118559.0

(51) Int. Cl. C07C 53/128; A61K 31/19; A61P 7/02

(52) U.S. Cl. CPC C07C 53/128; A61K 31/19; A61P 7/02

(57) ABSTRACT

The present invention relates to compounds of Formula I wherein R1 is either H or D and wherein D is deuterium, or a pharmaceutically acceptable salt thereof. The present invention also relates to methods of treating abnormal conditions associated with excess fibrin deposition, thrombus formation, migraine headaches, bipolar disorders, epilepsy and associated conditions in which inhibition of histone deacetylase (HDAC) provides a therapeutic benefit.

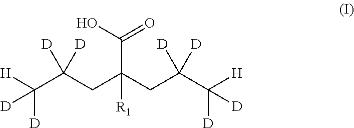

### FIELD OF THE INVENTION

The invention relates to novel compounds useful for treating abnormal conditions associated with excess thrombus formation, fibrin deposition, epilepsy, bipolar disease and/or histone deacetylation.

### BACKGROUND OF THE INVENTION

Valproic acid, which is commonly abbreviated VPA, is a well-known compound that was first used as an anticonvulsant to treat seizures, and it is also used to treat mania in patients with bipolar disorder and to prevent migraine headaches. In addition, VPA is an inhibitor of histone deacetylases (HDAC) and thus can alter gene expression. As such, VPA has recently been investigated as a potential anticancer therapeutic. It is not clear, however, if the ability of VPA to act as an HDAC inhibitor is related to its ability to treat seizures, bipolar disorders and prevent migraines.

Although administration of VPA may provide therapeutic benefits, there are significant toxicities that have been associated with VPA. Indeed, VPA administration can be associated with significant liver toxicity, including acute hepatic liver failure. In particular, there is mounting evidence that a common metabolite of VPA, 4-ene-VPA (depicted below) is at least partly responsible for the toxicity associated with VPA.

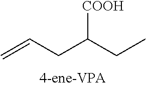

As with any active pharmaceutical ingredient, there may be that the risks associated with administering VPA can outweigh the benefits of administering this compound for any type of specific treatment. The problem plaguing the art, therefore, is that VPA, while beneficial, can provide too great of a toxicity risk to patients to be useful for use in treating certain patients. Thus, there is a need for new compounds and/or pharmaceutical compositions allowing for treatment of at least some of the medical indications mentioned above while minimizing side effects, such as liver toxicity.

### SUMMARY OF THE INVENTION

It is an object of the present disclosure to overcome or at least mitigate one or more of the aforementioned disadvantages. Further, it is an object of the present disclosure to provide advantages and aspects not provided by hitherto known techniques.

Thus, the present invention of the present disclosure provides at least one compound of Formula I

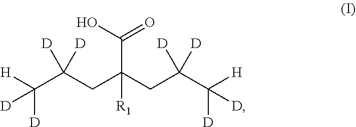

wherein R₁ is either H or D and wherein D is deuterium, or a pharmaceutically acceptable salt thereof. The present invention also relates to methods of treating abnormal conditions associated with excess fibrin deposition, thrombus formation, fibrosis, epilepsy, migraine headaches, bipolar disorders and conditions associated in which inhibition of histone deacetylase (HDAC) provides a therapeutic benefit.

### BRIEF DESCRIPTION OF THE DRAWINGS

FIG. 1 depicts the effects of bleeding time in mice treated with the compounds of the present invention. Tail bleeding time was assessed in mice treated with saline or 100 mg/kg of VPA, Compound Ia, Compound Ib, Rettie, A., et al., J. Biol. Chem., 263(27):13733-13738 (1988) Reference 4, '507 publication “D10” compound, or '507 publication “D11” compound and control. N=10 for control, VPA, Compound Ia, Compound Ib, D10 and D11. N=4 for Rettie, A., et al., J. Biol. Chem., 263(27):13733-13738 (1988) Reference 4 compound.

FIG. 2 depicts platelet accumulation in cremaster laser-induced thrombosis assay (30 mg/kg).

FIG. 3 depicts fibrin formation in cremaster laser-induced thrombosis assay (30 mg/kg).

FIG. 4 depicts platelet accumulation in cremaster laser-induced thrombosis assay (100 mg/kg).

FIG. 5 depicts formation of the 4-ene-VPA metabolite formation in VPA, Compound Ia (Cmpd Ia) and Compound Ib (Cmpd Ib). Values were normalized with the formation of the 4-ene-VPA metabolite in VPA set to 1.0.

### DETAILED DESCRIPTION OF THE INVENTION

The invention of the present disclosure relates at least one compound of Formula I

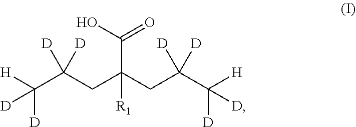

wherein R₁ is either H or D and wherein D is deuterium, or a pharmaceutically acceptable salt thereof. The phrase “compounds of the present invention” as used herein means any one or more of the specific compounds of Formula I.

In a particular embodiment, there is provided compounds of formula (I) where R₁ represents H.

In a further embodiment, there is provided compounds of formula (I) where R₁ represents D.

Specific compounds of the present invention include compounds Ia and/or Ib and pharmaceutically acceptable salts thereof:

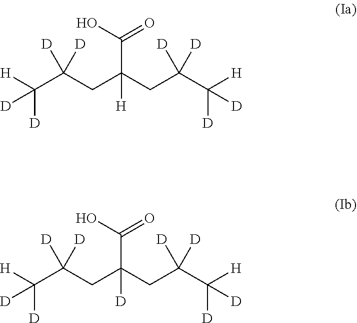 [SMILES: [2H]C([2H])C([2H])([2H])CC(CC([2H])([2H])C([2H])[2H])C(=O)O ; [2H]C([2H])C([2H])([2H])CC([2H])(CC([2H])([2H])C([2H])[2H])C(=O)O]

Compound Ia may be referred to herein as 2-(Propyl-2,2,3,3-d₄)pentanoic-4,4,5,5-d₄ acid, 2-[(2,2,3,3-2H4)propyl](4,4,5,5-2H4)pentanoic acid or 4,4,5,5-Tetradeutero-2-(2,2,3,3-tetradeuteropropyl)valeric acid (compound 1a) and compound Ib may be referred to herein as 2-(Propyl-2,2,3,3-d₄)pentanoic-2,4,4,5,5-d₅ acid or 2,4,4,5,5-Pentadeutero-2-(2,2,3,3-tetradeuteropropyl)valeric acid (compound 1b). The compounds of the present invention are novel derivatives of valproic acid (VPA), in which specific hydrogen atoms have been replaced with deuterium isotopes (²H) (represented as “D” in Formula I, compound Ia and compound Ib). The inventors have unexpectedly found that valproic acid having the specific deuteration patterns of Formula I have a surprising metabolic profile that reduces levels of a known toxic metabolite of VPA as well as increased safety profile that both treats conditions associated with excess fibrin deposition and/or thrombus formation as well as reducing excessive blood loss often seen in drugs targeting these conditions.

The terms “the compounds of the invention” and “a compound as described herein” are used interchangeably and can be used to indicate: the compound of Formula I, compound Ia, compound Ib.

Pharmacologically active compounds where hydrogen has been substituted with deuterium generally display the same pharmacodynamic effects as their non-deuterated counterparts. Deuteration, however, may alter metabolism of the parent compound in unpredictable ways. For example, Rettle, A., et a., J. Biol. Chem., 263(27):13733-13738 (1988) (“Rettie”) reported that deuteration of VPA in specific locations of VPA both reduces and increases the formation of the 4-ene-VPA metabolite. Specifically, Rettie notes that deuteration in the 4 and 4′-positions of VPA (4,4,4′,4′-D₄VPA), lead to considerably less formation of the 4-ene metabolite compared to VPA. On the other hand, Rettie reports that fully deuterated VPA at the 5 and 5′ positions (5,5,5,5′,5′,5′D₆VPA) formed an increased amount of the 4-ene-VPA metabolite, compared to VPA. These results would suggest that deuteration in the 4 and/or 4′ positions of VPA might reduce formation of the 4-ene metabolite and thus possibly reduce toxicity associated with VPA administration. But these results also suggest that deuteration in the 5 and 5′-positions of VPA would increase production of the 4-ene-VPA metabolite and thus increase toxicity associated with VPA administration. Rettie, therefore, would suggest that deuteration of the 5 and/or 5′ positions of VPA should be avoided.

The general concept of deuterated VPA is not new. For example, as noted above Rettie generated deuterated VPA compounds. In particular, Rettie generated four deuterated compounds: (4,4,-D₂VPA), (4,4,4′,4′-D₄VPA), (5,5,5-D₃VPA) (5,5,5,5′,5′,5′D₆VPA). Interestingly, the compounds deuterated at the 4 and 4′ positions produced less 4-ene-VPA than VPA in the metabolism studies, and the compounds deuterated at the 5 and 5′ positions produced more 4-ene-VPA than VPA in the metabolism studies. Again, these data would suggest the avoidance of deuterating the 5 and/or 5′ positions in VPA to reduce toxicity.

United States Pre-Grant Publication No. 2010/0143507, which was abandoned with no continuing applications filed thereon, also discloses a large genus of deuterated VPA compounds. The '507 publication, however, does not disclose or suggest any specific deuterated molecules having the specific pattern of deuteration of the compounds of the present invention. Moreover, the '507 publication does not provide any guidance or suggestions on which specific compounds produce less 4-ene-VPA when metabolized or which specific compounds may provide any therapeutic benefit. Indeed, the '507 publication does not contain any activity or metabolism data of any kind. And of the few specific molecules that the '507 publication discloses, the 5 and 5′ positions are either fully deuterated, i.e., the compounds have six (6) ²H isotopes at these two positions, or fully hydrogenated, i.e., the compounds have zero (0) ²H isotopes at these two positions.

United States Pre-Grant Publication No. 2012/0071554 (PCT Publication No. WO 2010/062656), which was abandoned with no continuing applications filed thereon, also discloses deuterated VPA compounds. The '554 publication, however, does not disclose or suggest any specific deuterated molecules having the specific pattern of deuteration of the compounds of the present invention. Moreover, the '554 publication does not provide any guidance or suggestions on which specific compounds produce less 4-ene-VPA when metabolized or which specific compounds may provide any therapeutic benefit. Indeed, the '554 publication does not contain any activity, stability or metabolism data of any kind. Of the few specific molecules that the '554 publication discloses, the 5 and 5′ positions are either fully hydrogenated, i.e., the compounds have zero (0) ²H isotopes at these two positions, deuterated at only one of these positions, i.e., the compounds have three (3) ²H isotopes at only one of these two positions, or are deuterated only once at each of these positions, i.e., the compounds have one (1) ²H isotope at each of these two positions.

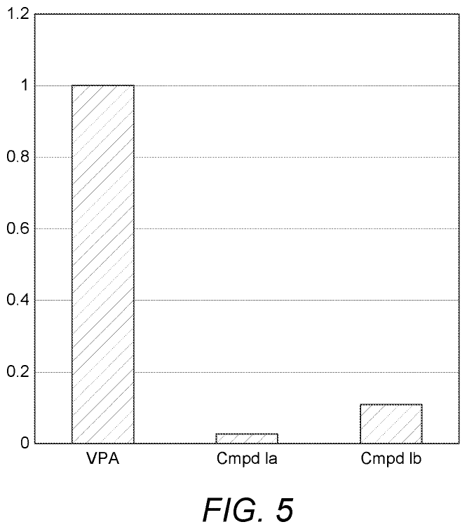

Thus, while the general concept of deuterated VPA may not be new, there is no guidance in the art that would suggest deuterating two hydrogens at each of the 5 and 5′ positions of VPA. In fact, Rettie, A., et al., J. Biol. Chem., 263(27):13733-13738 (1988)'s data would teach avoiding deuterating the 5 and/or 5′ positions of VPA altogether. The inventors, however, discovered that the compounds of the present invention, which are deuterated 4 times at the 5 and 5′ positions, i.e., the compounds have two (2) ²H isotopes at each these two positions, have a drastic reduction in metabolism to the toxic 4-ene-VPA metabolite. Specifically, formation of the 4-ene-VPA metabolite was reduced by approximately 97% for compound Ia and approximately 89% for compound Ib when compared with VPA, as provided in Example 4 and FIG. 5. This drastic reduction in formation of the toxic metabolite from the compounds of the present invention was not predictable. Accordingly, the present invention provides novel, safer compounds for treating conditions for which deuterated VPA is or could be used as a monotherapy or part of a combination therapy. As noted below, the compounds of the present invention also have unexpectedly superior therapeutic advantages over VPA.

Surprisingly, the compounds described herein, which are novel derivatives of VPA with specific deuteration patterns, not only reduces formation of the toxic 4-ene-VPA metabolite, but may also provide an unexpectedly superior performance in reducing excess bleeding when administered compared to VPA. Moreover, these compounds provide advantageous results regarding platelet activation and fibrin formation over VPA, indicating that the compounds are useful in treating conditions associated with excess thrombus formation and/or fibrin deposition.

Accordingly, the invention also provides methods of using the compounds of the present invention for treating abnormal conditions associated with thrombus formation, excess fibrin deposition, and/or fibrosis. In other words, the present invention provides methods of treating abnormal conditions associated with thrombus formation, excess fibrin deposition and/or fibrosis in subjects in need of treatment thereof. The methods of treatment of abnormal conditions associated with thrombus formation, excess fibrin deposition and/or fibrosis comprise administering a therapeutically effective amount of one or more of the compounds of the present invention to a subject in need of treatment thereof.

There is also provided a compound as described herein, i.e., a compound of the invention, or a pharmaceutical composition as described herein, for use in the treatment and/or prevention of an abnormal condition associated with thrombus formation, excess fibrin deposition and/or fibrosis. Administering a pharmaceutical composition as described herein may reduce excessive blood loss in the subject being treated. Thus, methods of treating or preventing conditions marked or associated with thrombus formation, excess fibrin deposition and/or fibrosis as described herein (and compounds for use in such methods) may also involve reducing excessive blood loss in the subject being treated.

There is also provided use of a compound as described herein, or a pharmaceutical composition as described herein, for the manufacture of a medicament for the treatment of an abnormal condition associated with thrombus formation, excess fibrin deposition and/or fibrosis.

According to an aspect of the invention there is thus provided a compound of the invention for use as a medicament.

Examples of conditions marked or associated with thrombus formation and/or excess fibrin deposition for which one or more of the compounds of the present invention may be used to treat and/or prevent in a subject include but are not limited to atherosclerosis, myocardial infarction, ischemic stroke, deep vein thrombosis, superficial vein thrombosis, thrombophlebitis, pulmonary embolism, disseminated intravascular coagulation, renal vascular disease and intermittent claudication. In one embodiment, the condition is selected from the group consisting of myocardial infarction, ischemic stroke, deep vein thrombosis and pulmonary embolism. Particular pathological conditions associated with fibrosis, for which the compounds of the present invention may be useful in treating and/or preventing include but are not limited to cardiac fibrosis, such as myocardial fibrosis, atrial fibrosis, fibrosis associated with cardiac hypertrophy and remodelling, fibrosis associated with myocardial infarction, atrial fibrillation and heart failure, right and left heart failure with reduced as well as preserved ejection fraction, cardiac fibrosis associated with lung disease, fibrosis associated with pulmonary arterial hypertension, fibrosis associated with thromboembolic disease, fibrosis associated with NASH, liver cirrhosis and portal hypertension, kidney fibrosis with and without systemic hypertension, pulmonary fibrosis (e.g. idiopathic pulmonary fibrosis), peritoneal fibrosis, i.e. progressive fibrous thickening of the peritoneal membrane, conjunctival fibrosis, i.e., overproduction of collagen type I in the membranes of the inner surfaces of the eyelids and the front of the eyeball, and/or renal fibrosis associated with chronic kidney disease.

The methods of using the compounds of the present invention for treating abnormal conditions associated with thrombus formation, excess fibrin deposition, and/or fibrosis may reduce excessive blood loss in the subject.

As described herein, several conditions and risk factors are associated with increased susceptibility to thrombotic events (i.e. thrombus formation). These include atherosclerosis, hypertension, abdominal obesity, smoking, sedentary lifestyle, and low-grade inflammation. Thus, in particular embodiments of the invention, the treatment or prevention is in a patient having one or more such condition/risk factor.

In more particular embodiments, the patient at increased risk of developing a pathological condition associated with excess fibrin deposition and/or thrombus formation is a patient who:
(i) is suffering from one or more medical condition associated with increased risk of thrombus formation, such as metabolic syndrome (e.g. type H diabetes), oncologic diseases, heart failure, renal failure and/or sepsis;
(ii) has previously experienced one or more incidence of a pathological condition associated with excess fibrin deposition and/or thrombus formation, such as one or more incidence of myocardial infarction, ischemic stroke and pulmonary embolism (e.g. one or more incidence of ischemic stroke, such as a major ischemic stroke, minor ischemic stroke or TIA); and/or
(iii) has one or more lifestyle and/or environmental factors placing them at said increased risk, such the patient being a smoker, obese and/or having decreased mobility (e.g. the patient is bed-ridden, such as a patient in a medical unit or elderly care unit).

In particular embodiments of the invention, pathological conditions that may be treated or prevented in accordance with the invention are those that are caused wholly or at least in part by an increased fibrin deposition and/or reduced fibrinolytic capacity due to local or systemic inflammation. These include, but are not limited to, myocardial infarction, stable angina pectoris, unstable angina pectoris, intermittent claudication, ischemic stroke, transient ischemic attack, deep vein thrombosis and pulmonary embolism.

Particular biomarkers that may identify local or systemic inflammation include high sensitive C-reactive protein (hs-CRP) (at or above 2.0 mg/I serum) and fibrinogen (at or above 3 g/l serum) (Corrado E., et al. An update on the role of markers of inflammation in atherosclerosis, Journal of Atherosclerosis and Thrombosis, 2010; 17:1-11, Koenig W., Fibrin(ogen) in cardiovascular disease: an update, Thrombosis Haemostasis 2003; 89:601-9).

In one embodiment, the treatment or prevention includes reversal and prevention of fibrosis. In more specific embodiments, the treatment or prevention includes reversal and prevention of either primary or secondary fibrosis or both, remodelling and repair as well as effects on fibrosis associated with cardiovascular disease, inflammatory disease, fibrosis associated with activation of the Renin-Angiotensin-System, the mineral corticoid receptor and PAI-1 as well as other systemic diseases.

In another embodiment the fibrosis is selected from the group consisting of cardiac fibrosis, arterial fibrosis, pulmonary fibrosis, fibrosis associated with pulmonary arterial hypertension, fibrosis associated with thromboembolic disease, fibrosis associated with NASH, kidney fibrosis, eye fibrosis, skin fibrosis, liver fibrosis, pancreas fibrosis and other GI tract fibrosis.

In one embodiment, the one or more of the compounds of the present invention may be used to treat and/or prevent diseases associated with pulmonary or systemic increased blood pressure.

In one embodiment, the one or more of the compounds of the present invention may be used to treat and/or prevent pulmonary arterial hypertension (PAH) and/or chronic thromboembolic pulmonary hypertension (CTEPH).

In one embodiment, the one or more of the compounds of the present invention may be used to treat and/or prevent thrombus formation associated with increased pulmonary pressure, including but not limited to, pulmonary arterial hypertension (PAH) and/or chronic thromboembolic pulmonary hypertension (CTEPH).

The invention also provides methods of using the compounds of the present invention to inhibit histone deacetylase. In other words, the present invention provides methods of inhibit histone deacetylase in subjects in need of inhibition thereof. The methods of inhibiting histone deacetylase comprise administering a therapeutically effective amount of one or more of the compounds of the present invention to a subject in need of inhibition of histone deacetylase thereof.

There is also provided use of a compound as described herein, or a pharmaceutical composition as described herein, for the manufacture of a medicament for the inhibition of histone deacetylase.

There is also provided a compound as described herein, or a pharmaceutical composition as described herein for use in the inhibition of histone deacetylase.

There is also provided the use of a compound as described herein, or a pharmaceutical composition as described herein, in the inhibition of histone deacetylase. In particular embodiments, such use may be described as non-therapeutic and/or ex vivo.

The invention also provides methods of using the compounds of the present invention to treat a subject diagnosed as having bipolar disorder. In other words, the present invention provides methods of treating bipolar disorder in a subject in need of treatment thereof. The methods of treatment of bipolar disorder comprise administering a therapeutically effective amount of one or more of the compounds of the present invention to a subject in need of treatment thereof.

There is also provided a compound as described herein, or a pharmaceutical composition as described herein for use in the treatment and/or prevention of bipolar disorder.

There is also provided use of a compound as described herein, or a pharmaceutical composition as described herein, for the manufacture of a medicament for use in the treatment and/or prevention of bipolar disorder.

The invention also provides methods of using the compounds of the present invention to treat a subject diagnosed as having epilepsy. In other words, the present invention provides methods of treating epilepsy in a subject in need of treatment thereof. The methods of treatment of epilepsy comprise administering a therapeutically effective amount of one or more of the compounds of the present invention to a subject in need of treatment thereof.

There is also provided a compound as described herein, or a pharmaceutical composition as described herein, for use in the treatment and/or prevention of epilepsy.

There is also provided use of a compound as described herein, or a pharmaceutical composition as described herein, for the manufacture of a medicament for use in the treatment and/or prevention of epilepsy.

For the avoidance of doubt, the methods of treatment of epilepsy comprise administering a therapeutically effective amount of one or more of the compounds of the present invention to a subject in need of treatment thereof.

The invention also provides methods of using the compounds of the present invention to prevent a migraine headache in a subject. In other words, the present invention provides methods of reducing the likelihood or reducing the frequency of migraine headaches in a subject in need of reduction thereof. The methods of treatment of a migraine headache comprise administering a therapeutically effective amount of one or more of the compounds of the present invention to a subject in need of treatment thereof.

There is also provided a compound as described herein, or a pharmaceutical composition as described herein, for use in the treatment and/or prevention of a migraine headache.

There is also provided use of a compound as described herein, or a pharmaceutical composition as described herein, for the manufacture of a medicament for use in the treatment and/or prevention of a migraine headache.

The methods of treatment and methods using the compounds of the present invention comprise administering one of more the compounds of the present invention to a subject in need of treatment thereof. Suitable dosage ranges of the compounds of the invention are generally about 0.0001 milligrams/dose to 2000 milligrams/dose of a compound of the invention per kilogram body weight, per day. In specific embodiments of the invention, the dose is from about 0.001 milligram to about 4000 milligrams per kilogram body weight, or from about 0.01 milligram to about 3000 milligrams per kilogram body weight, or from about 0.1 milligram to about 2000 milligrams per kilogram body weight, or from about 0.1 milligram to about 1500 milligrams per kilogram body weight, or from about 0.1 milligram to about 1000 milligrams per kilogram body weight, or from about 1 milligram to about 500 milligrams per kilogram body weight, or from about 1 milligram to about 100 milligrams per kilogram body weight, or from about 1 milligram to about 90 milligrams per kilogram body weight, or from about 1 milligram to about 80 milligrams per kilogram body weight, or from about 1 milligram to about 70 milligrams per kilogram body weight, or from about 1 milligram to about 60 milligrams per kilogram body weight, or from about 1 milligram to about 50 milligrams per kilogram body weight, or from about 1 milligram to about 40 milligrams per kilogram body weight, or from about 1 milligram to about 30 milligrams per kilogram body weight, or from about 1 milligram to about 20 milligrams per kilogram body weight, or from about 1 milligram to about 10 milligrams per kilogram body weight, or from about 1 milligram to about 5 Milligrams per kilogram body weight. In other embodiments, one of more of the compounds of the present invention are administered at a dose of about 1 mg/kg, 2 mg/kg, 3 mg/kg, 5 mg/kg, 7 mg/kg, 10 mg/kg, 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65/kg, 70 mg/kg, 75 mg/kg, 80 mg/kg, 85 mg/kg, 90 mg/kg, 95 mg/kg or 100 mg/kg. In other specific embodiments, daily doses are in the range of about 1 to 4000 mg per patient (e.g. 1 to 3000 mg or 1 to 2000 mg per patient), administered in single or multiple doses. In other more specific embodiments, one of more of the compounds of the present invention are administered at a daily dose of about 10 mg to about 2000 mg, from about 50 mg to about 1300 mg, e.g., about 100 mg to about 1200 mg, or from about 50 mg to about 1000 mg, e.g., about 100 mg to about 800 mg, about 100 mg to about 600 mg, or about 200 mg to about 600 mg, e.g., about 100 mg to about 800 mg, or about 200 mg to about 600 mg. The daily doses may be administered as a single bolus dose or the total dose may be divided over multiple doses, e.g., 2, 3, 4, 5, 6, 7 or 8 doses, per day.

The compounds of the present invention include pharmaceutically acceptable salts of the compounds of Formula I. The phrase “pharmaceutically acceptable salt(s),” as used herein includes but is not limited to salts of acidic or basic groups that may be present in compounds used in the present compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions including, but not limited to, sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that include an amino moiety may form pharmaceutically acceptable salts with various amino acids, in addition to the acids mentioned above. The compounds described herein are acidic in nature and are capable of forming salts with e.g. various pharmacologically acceptable cations. Examples of such salts include alkali metal or alkaline earth metal salts and, particularly, calcium, magnesium, sodium, lithium, zinc, potassium, and iron salts. Particular examples of pharmaceutically acceptable addition salts include those derived from metals such as calcium, magnesium, potassium or, preferably sodium. In certain embodiments, such salts may be present as a “hemi-salt” (i.e. in a 2:1 ratio of compound to counterion). The compounds described herein may also for complexes with various amines. Examples of amines include, alkylamines, aminoalcohols (e.g. 2-(dimethylamino)ethanol), basic amino acids (e.g. lysine), quartenary amines (e.g. choline).

As used herein and unless otherwise indicated, the phrase “therapeutically effective amount” of a composition of the invention is measured by the therapeutic effectiveness of a compound of the invention, wherein at least one adverse effect of a disorder is ameliorated or alleviated.

The terms “preventing” or “prevention” is intended to include reducing the frequency or likelihood of (e.g. reducing the risk of) a subject experiencing an undesired physiological activity or symptom associated with an abnormal condition or disorder. The term “prevent” or “prevention” as use herein does not require absolute prevention of the abnormal condition. In one embodiment, “treatment” or “treating” refers to reducing or amelioration of a disease, disorder, abnormal condition or at least one discernible symptom thereof. In another embodiment, “treatment” or “treating” refers to an amelioration of at least one measurable physical parameter, not necessarily discernible by the patient. In yet another embodiment, “treatment” or “treating” refers to inhibiting the progression of a disease or disorder, either physically, e.g., stabilization of a discernible symptom, physiologically, e.g., stabilization of a physical parameter, or both. In yet another embodiment, “treatment” or “treating” refers to delaying the onset of a disease, disorder or abnormal condition.

As used herein, the skilled person will understand that references to “prevent” or “prevention” of a particular condition may also be referred to as “prophylaxis” of said condition, and vice versa. Thus, each reference herein to “preventing” a condition may be replaced with a reference to “prophylaxis” of said condition.

In certain embodiments, the compositions of the invention are administered to a patient, for example a human. The terms subject and patient are used interchangeably herein. The subject can be a non-human mammal as well, e.g., for veterinary use for companion pets or for farming or livestock animals. Examples of non-human subject include but are not limited to non-human primates, dogs, cats, cows, pigs, oxen, horses, etc.

The compounds of the invention may be administered by any convenient or conventional route, for example, oral, by infusion or bolus injection, by absorption through epithelial or mucocutaneous linings, e.g., oral mucosa, rectal and intestinal mucosa, etc., and may be administered together with another biologically active agent. Administration can be systemic or local. Various delivery systems are known, e.g., encapsulation in liposomes, microparticles, microcapsules, capsules, etc., and can be used to administer a compound or composition of the invention. Methods of administration include but are not limited to intradermal, intramuscular, intraperitoneal, intravenous, subcutaneous, intranasal, epidural, oral, sublingual, intranasal, intracerebral, intravaginal, transdermal, rectally or topically, for example to the ears, nose, eyes, or skin. In a specific embodiment, the compounds of the invention are administered orally.

In specific embodiments, it may be desirable to administer one or more compounds of the invention locally to the area in need of treatment. This may be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In another embodiment, the compounds of the invention can be delivered in a vesicle, for example a liposome. See Langer, Science 249:1527-1533 (1990); Treat et al., in Liposomes in the Therapy of infectious Disease and Cancer, Lopez-Berestein and Fidler (eds.), Liss, New York, pp. 353-365 (1989).

In yet another embodiment, the compounds of the invention can be delivered in a controlled release system. In one embodiment, a pump may be used. See Sefton, 1987, CRC Crit. Ref. Biomed. Eng. 14:201; Buchwald et al., 1980, Surgery 88:507 Saudek et al., 1989, N. Engl. J Med. 321:574. In another embodiment, polymeric materials can be used. See Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

All formulations known in the art and described for valproic acid, and pharmaceutically acceptable salts thereof, may be used when administering the compounds of the present invention. For example, in known pharmaceutical formulation comprising valproic acid in admixture with a pharmaceutically acceptable adjuvant, diluent or carrier, the compositions and methods of the present invention contemplate substituting VPA in these known formulations with one or more of the compounds of the present invention.

The present compositions will contain a therapeutically effective amount of a compound of the invention, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which a compound of the invention is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used. Water can be a vehicle when the compound of the invention is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable pharmaceutical vehicles are described in “Remington's Pharmaceutical Sciences” by A. R. Gennaro. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use.

In another embodiment, the compounds of the invention are formulated in accordance with routine procedures as a pharmaceutical composition adapted for intravenous administration to humans. Typically, compounds of the invention for intravenous administration are solutions in sterile isotonic aqueous buffer. Where necessary, the compositions may also include a solubilizing agent. Compositions for intravenous administration may optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients may be mixed prior to administration.

Formulations for oral delivery may be in the form of tablets, lozenges, aqueous or oily suspensions, granules, powders, emulsions, capsules, syrups, or elixirs, for example. Orally administered compositions may contain one or more optionally agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions may be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate may also be used. Oral compositions can include standard vehicles such as mannitol, lactose, starch, magnesium stearate, sodium saccharine, cellulose, magnesium carbonate, etc.

The skilled person will understand that compounds of formula (I) (including compounds of formula (Ia) and (Ib)) may be prepared using techniques known to those skilled in the art.

In a particular embodiment, there is a process for the preparation of a compound of formula (Ia) or a compound of formula (Ib),

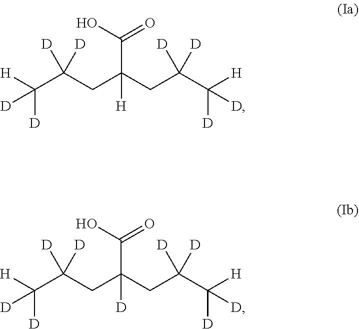

or a pharmaceutically acceptable salt thereof, comprising the step of decarboxylation of a compound of formula (II),

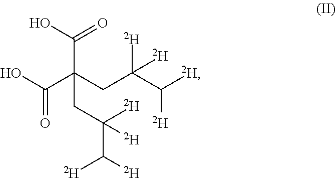

under conditions known to those skilled in the art (such as by heating to a suitable temperature for a suitable period of time, e.g. heating to 160° C. over night) optionally in the presence of D₂O,
wherein reaction in the absence of D₂O results in preparation of a compound of formula (Ia) and reaction in the presence of D₂O (e.g. in the presence of an excess of D₂O, such as wherein D₂O is used a solvent) results in the preparation of a compound of formula (Ib),
and, where the compound of formula (Ia) or (Ib) is in the form of a pharmaceutically acceptable salt, optionally comprising the further step of reacting with a suitable salt counterion (i.e. reacting with a compound suitable for forming the salt, such as a suitable base, e.g. wherein the salt is a sodium salt or hemi sodium salt, a suitable amount of sodium hydroxide).

The skilled person will also understand that starting materials used in the preparation of compounds of formula I may be prepared using techniques known to those skilled in the art.

In a particular embodiment, there is provided a process for preparing a compound of formula (II) comprising the step of hydrolysis of a compound of formula (III),

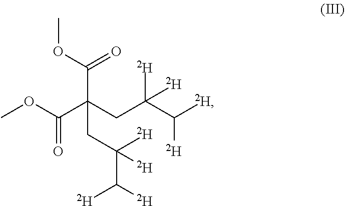

under conditions known to those skilled in the art (such as by hydrolysis in the presence of a suitable base, e.g. an aqueous base, such as aqueous sodium hydroxide).

In a particular embodiment, there is provided a process for the preparation of a compound of formula (III) comprising the step of hydrogenation of a compound of formula (IV),

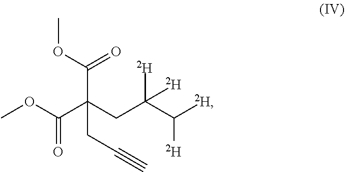

under conditions known to those skilled in the art (such as in the presence of a suitable catalyst, e.g. a rhodium catalyst) and the presence of a suitable source of deuterium (e.g. deuterium gas).

In a particular embodiment, there is provided a process for the preparation of a compound of formula (IV) comprising the step of alkylation of a compound of formula (V),

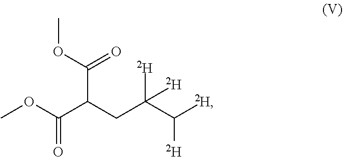

under conditions known to those skilled in the art, such as by reaction with a suitable base metal (e.g. a metal hydride, such a sodium hydride) followed by reaction with a suitable alkylating agent (e.g. a suitable alkyl halide, such as propargyl bromide).

In particular embodiment, there is provided a process for preparing a compound of formula (I) (i.e. a compound of formula (Ia) or Ib)) comprising:
a process for preparing a compound for formula (IV) as described herein;
followed by a process for preparing a compound for formula (III) as described herein;
followed by a process for preparing a compound for formula (II) as described herein;
followed by a process for preparing a compound for formula (I) as described herein.

The skilled person will understand that compounds of formula (II), (III). (IV) and (V) may be novel. Thus, there is also provided herein a compound of formula (II), (III). (IV) and/or (V), and pharmaceutically acceptable salts thereof.

Compounds of formula (V) may be commercially available and/or may be synthesized using techniques known to those skilled in the art.

Such compounds may be isolated from their reaction mixtures and, if necessary, purified using conventional techniques as known to those skilled in the art. Thus, processes for preparation of compounds of the invention as described herein may include, as a final step, isolation and optionally purification of the compound of the invention (e.g. isolation and optionally purification of the compound of formula I).

### EXAMPLES

The present invention will be further described by reference to the following examples, which are not intended to limit the scope of the invention.

In the event that there is a discrepancy between nomenclature and any compounds depicted graphically, then it is the latter that presides (unless contradicted by any experimental details that may be given or unless it is clear from the context).

### Example 1—Synthetic Routes

### Synthesis of Compound Ia and Compound Ib

### Step 1.

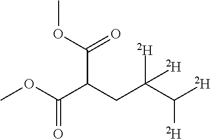 [SMILES: [2H]C([2H])C([2H])([2H])CC(C(=O)OC)C(=O)OC]

### Dimethyl 2-(2,2,3,3-tetradeuteriopropyl)propanedioate

Dimethyl propargylmalonate (10.0 g, 58.8 mmol) and tris(triphenylphosphine)rhodium(I) chloride (1.6 g, 1.8 mmol, 3%) were mixed in 100 ml of toluene. The reaction flask was evacuated and flushed with nitrogen repeatedly. The reaction flask was connected to the hydrogenation manifold and the reaction flask evacuated and flushed with deuterium. The mixture was stirred under deuterium. After complete reduction the reaction mixture was filtered through celite and washed with water and brine. The organic phase was dried over MgSO₄, filtered and concentrated. The residue was purified by kugelrohr distillation (10 mbar@130° C.). A clear, colorless oil was obtained (7.2 g, 69%), ¹H NMR (400 MHz, Chloroform-d) δ 3.73 (s, 6H), 3.37 (t, J=7.6 Hz, 1H), 1.87 (d, J=7.6 Hz, 2H), 0.88 (s, 1H).

### Step 2.

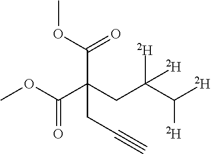 [SMILES: [2H]C([2H])C([2H])([2H])CC(CC#C)(C(=O)OC)C(=O)OC]

### Dimethyl 2-prop-2-ynyl-2-(2,2,3,3-tetradeuteriopropyl)propanedioate

Sodium hydride (60% in mineral oil, 5.55 g, 139 mmol) was slurried in 200 ml of THF cooled on ice under nitrogen. Dimethyl 2-(2,2,3,3-tetradeuteriopropyl)propanedioate (16.5 g, 92.6 mmol) in 50 ml of THF was added dropwise. After 30 minutes propargyl bromide (14.3 g, 120 mmol) in 50 ml of THF was added dropwise. The reaction mixture was stirred for 2 hours at 0° C. and quenched by the addition of 100 ml of sat. NH₄Cl. Heptane (200 ml) was added, and the phases separated. The organic phase was washed with sat. NaHCO₃ and brine, dried over MgSO₄, filtered and concentrated. A light brown oil was obtained (18 g). Crude, contains mineral oil, used as such, ¹H NMR (400 MHz, Chloroform-d) δ 3.74 (s, 6H), 2.82 (d, J==2.7 Hz, 2H), 2.05-1.96 (m, 3H), 0.89 (s, 1H).

### Step 3.

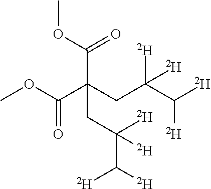 [SMILES: [2H]C([2H])C([2H])([2H])CC(CC([2H])([2H])C([2H])[2H])(C(=O)OC)C(=O)OC]

### Dimethyl 2,2-bis(2,2,3,3-tetradeuteriopropyl)propanedioate

Dimethyl 2-prop-2-ynyl-2-(2,2,3,3-tetradeuteriopropyl)propanedioate (18 g, 83 mmol) and tris(triphenylphosphine)rhodium(I) chloride (1.54 g, 1.66 mmol, 2%) were mixed in 300 ml of toluene. The reaction flask was evacuated and flushed with nitrogen repeatedly. The reaction flask was connected to the hydrogenation manifold and the reaction flask evacuated and flushed with deuterium. The mixture was stirred under deuterium. After 1, 2 and 3 days, 0.5 g more of rhodium catalyst was added and the reaction restarted. After complete reduction the solvent was removed under reduced pressure. Heptane (200 ml) was added. After stirring for 30 minutes the precipitate was removed by filtration through celite. The mother liquor was concentrated, and the residue purified by kugelrohr distillation (30 mbar@160° C.). A clear, colourless oil was obtained (13 g, 70%), 1H NMR (400 MHz, Chloroform-d) δ 3.70 (s, 6H), 1.83 (s, 4H), 0.86 (s, 2H).

### Step 4.

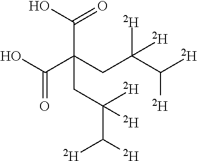 [SMILES: [2H]C([2H])C([2H])([2H])CC(CC([2H])([2H])C([2H])[2H])(C(=O)O)C(=O)O]

### 2,2-Bis(2,2,3,3-tetradeuteriopropyl)propanedioic acid

Sodium hydroxide (13.9 g, 348 mmol) was dissolved in 100 ml of water. Dimethyl 2,2-bis(2,2,3,3-tetradeuteriopropyl)propanedioate (13 g, 58 mmol) in 50 ml of methanol was added. The reaction mixture was stirred at reflux for 5 hours and at room temperature overnight. The reaction mixture was washed with 2*50 ml of DCM. The aqueous phase was concentrated under vacuum to remove traces of DCM. The solution was cooled on ice and 80 ml of 5 M HCl was added. A white precipitate formed. The mixture was stirred on ice for 1 hour and the precipitate collected by filtration and washed with a small amount of water. White solid (11 g, 97%), ¹H NMR (500 MHz, Chloroform-d) δ 1.92 (s, 4H), 0.88 (s, 2H).

### Step 5.

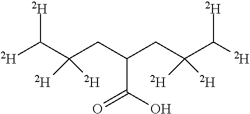 [SMILES: [2H]C([2H])C([2H])([2H])CC(CC([2H])([2H])C([2H])[2H])C(=O)O]

### “Compound Ia”, 2-[(2,2,3,3-2H4)propyl](4,4,5,5-2H4)pentanoic acid

2,2-Bis(2,2,3,3-tetradeuteriopropyl)propanedioic acid (11 g, 56 mmol) was mixed with 100 ml of water in a glass insert to a steel bomb. The bomb was heated to 160° C. over night. After cooling to room temperature, the water/oil mixture was transferred to a separation flask with heptane. The aqueous phase was extracted three times with heptane. The combined organic phases were washed with brine, dried over Na₂SO₄, filtered and concentrated to a light brown oil (8.0 g, 94%). Contains traces of heptane, ¹H NMR (400 MHz, Chloroform-d) δ 2.42-2.30 (m, 1H), 1.60 (dd, J=13.4, 8.9 Hz, 2H), 1.43 (dd, J=13.4, 5.3 Hz, 21H), 0.86 (s, 21H).

### Step 6.

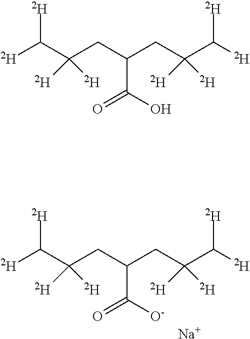 [SMILES: [2H]C([2H])C([2H])([2H])CC(CC([2H])([2H])C([2H])[2H])C(=O)O ; [2H]C([2H])C([2H])([2H])CC(CC([2H])([2H])C([2H])[2H])C(=O)[O-] ; [Na+]]

### “Compound Ia, sodium hemi salt”, 2-[(2,2,3,3-2H4)propyl](4,4,5,5-2H4)pentanoic acid and sodium 2-[(2,2,3,3-2H4)propyl](4,4,5,5-2H4)pentanoate

Compound Ia (8.00 g, 52.5 mmol) and finely ground sodium hydroxide (1.05 g, 26.3 mmol) were mixed in 20 ml of MTBE. The mixture was stirred at 50° C. for 30 minutes. A Clear, light brown solution was obtained. After cooling on ice, 80 ml of acetonitrile was added. A massive precipitate formed. After stirring for one hour on ice bath the precipitate was collected by filtration and washed with acetonitrile. The solid was dried under vacuum overnight. White solid (6.79 g, 79%, assumed hemi salt), ¹H NMR (400 MHz, DMSO-d6) δ 2.06 (ddd, J=14.1, 9.0, 5.2 Hz, 1H), 1.41 (dd, J=12.9, 9.0 Hz, 2H), 1.19 (dd, J=13.0, 5.2 Hz, 2H), 0.77 (s, 2H), LCMS (ESI−): m/z [M-H]− calcd.: 151, found: 151

Compound Ib was prepared as described above but with the addition of performing the final decarboxylation in D₂O.

### Example 2—Excessive Bleeding Assay

To determine the risks of excessive bleeding associated with the compounds of the present invention, the last 5 mm of the tail of mice was resected to identify the amount of time necessary for the vessel to properly clot and prevent excessive loss of blood. Briefly, mice were administered Compound Ia (Formula Ia), Compound Ib (Formula Ib), “²H₄-VPA” from Rettie, A., et al, J. Biol. Chem., 263(27):13733-13738 (1988) (“Reference 4” compound), the compound of claim 11 from the US 2010/0143507 publication (“D11”), the compound of claim 12 from the US 2010/0143507 publication (“D10”), VPA or vehicle control (sodium chloride saline) and a bleeding time assay was conducted.

10-12 weeks old C57BL/6 wild type mice were purchased from Jackson Laboratories for these experiments. Various compounds were administered to the mice via IP injection once a day at a dose of 100 mg/kg of compound for five days prior to resection.

Mice were sedated with 250 μL of ketamine solution (Ketamine (100 μL)/xylazine (50 μL) in 850 μl. Saline solution). The mice were then placed face down on a heat pad on top of the bead bath to enable the tail to be pulled into a saline-filled tube. 5 mm of the tail as cut with a scalpel and the tail was placed in a saline-filled tube and a timer started. The timer was stopped when the bleeding stopped, but the possibility of re-bleeds was observed from 10 minutes after initial bleeding stopped. If the mouse bleeds continuously for 10 minutes, the experiment was halted and the mouse was euthanized. One-way ANOVA with Dunnett's correction for multiple comparison test was performed to determine statistical significance of bleeding times.

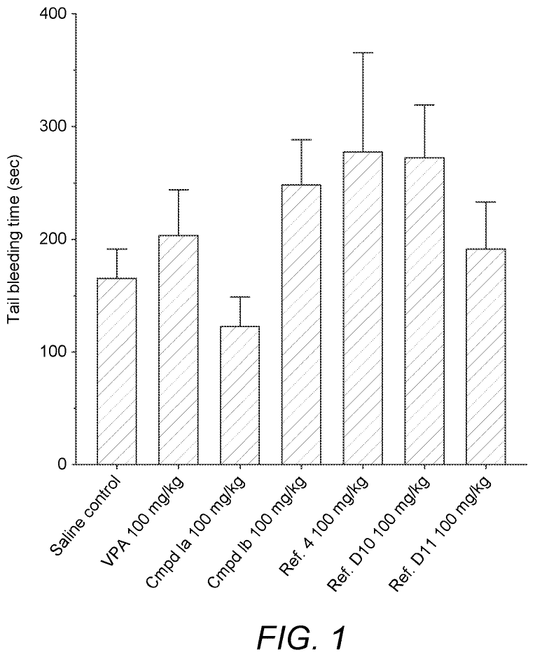

As shown in FIG. 1, bleeding time for the control mice were approximately 100 seconds. Interestingly, 100 mg/kg of VPA and Reference D10 resulted in a significant increase in bleeding time compared to control. Treatment with all compounds, except for compound Ia, resulted in increased bleeding time, compared with control time. Of note, the Reference 4 compound from Rettie, A., et al., J. Biol. Chem., 263(27):13733-13738 (1988) displayed markedly higher bleeding times than compound Ia. Taken together, this data shows that Compound Ia has the best safety profile in terms of risk of excess bleeding of all compounds tested.

### Example 3—Thrombosis Assessment of Compounds of the Invention

The effects of the compounds of the invention and VPA on the ability and timing of clot formation were determined. Specifically, platelet accumulation and fibrin formation in the cremaster artery in forming a non-occlusive clot were measured following laser-induced insult to the vessel in 10-12 week-old mice (C57BL/6 wild type mice purchased from Jackson Laboratories).

Valproic acid (VPA) was purchased from Sigma-Aldrich as a powder and resuspended in 0.9% Sodium Chloride (saline) to prepare a final stock solution of each compound. The VPA and other compounds (compound Ia and compound Ib) were administered via IP injection to the mice at a final concentration of 30 mg/kg and 100 mg/kg.

To assess platelet accumulation and fibrin formation, a Zeiss Axio Examiner Z1 fluorescent multichannel intravital microscope was used. The microscope was equipped with: (1) a solid laser launch system (LaserStack; Ablate! Photoablation system, intelligent Imaging innovations), (2) a high-speed sCMOS camera, and (3) a laser ablation system (intelligent Imaging innovations, Denver, CO, USA). SlideBook 6.0 digital microscopy software for image recording and analysis was also used. An anti-platelet antibody, and an anti-fibrin antibody, both commercially available, were used for imaging purposes.

The dynamic accumulation of platelets and fibrin within thrombi at the site of the injury in vivo was evaluated in cremaster arteriole in response to laser-induced injury under intravital microscopy. Adult male mice (10-12 weeks old) were treated with 30 mg/kg and 100 mg/kg for 5 days prior to injury were anesthetized by an intraperitoneal injection of ketamine/xylazine (100 and 10 mg/kg, respectively) and the jugular vein was cannulated. A tracheal tube was inserted to facilitate breathing. The cremaster arteriole was surgically prepared and superfused with preheated bicarbonate saline buffer throughout the experiment.

DyLight 488-conjugated rat anti-mouse platelet GP1bβ antibody (0.1 μg/g; EMFRET Analytics) and Alexa Fluor 647-conjugated anti-fibrin (0.3 μg/g) were administered intravenously via a jugular vein cannula prior to vascular injury. Microcirculation was monitored and recorded under multichannel intravital microscopy.

Multiple independent thrombi (6-8 thrombi in each mouse) were induced in the arterioles (30-50 μm diameter) in each mouse by a laser ablation system. Images of thrombus formation at the site of injured arterioles were acquired in realtime under 63× water-immersion objective with a Zeiss Axio Examiner Z1 fluorescent microscope equipped with solid laser launch system (LaserStack; Intelligent Imaging Innovations) and high-speed sCMOS camera. All captured images were analyzed for the change of fluorescent intensity over the course of thrombus formation after subtracting fluorescent background using the Slidebook program. A one way ANOVA with Dunnett's testing for multiple comparison was performed to assess statistical significance.

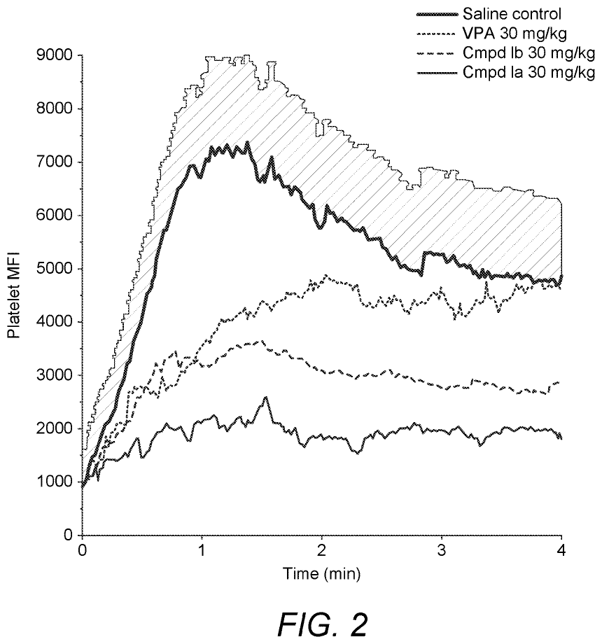

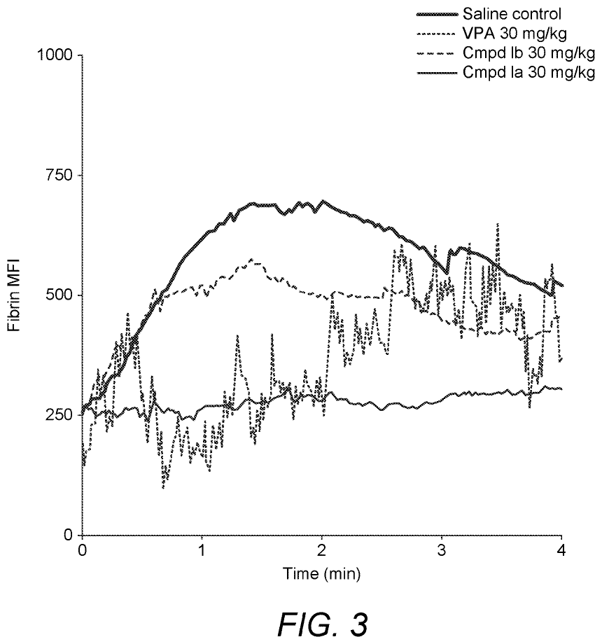

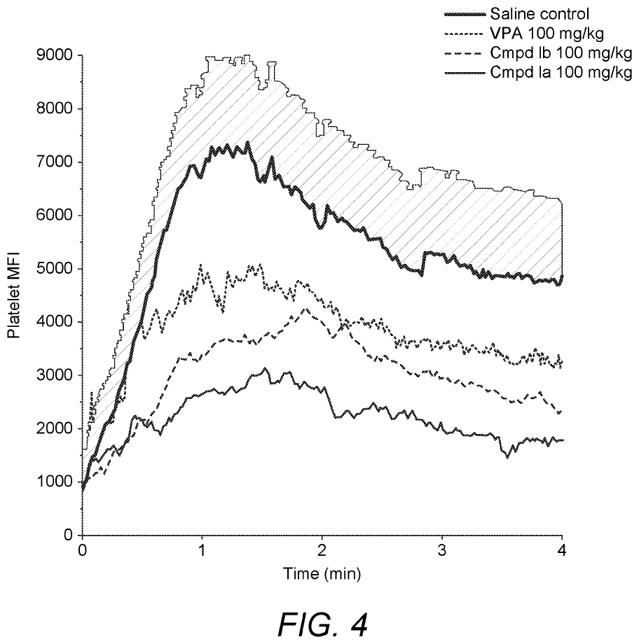

As shown in FIGS. 2 and 3 compound Ia at a dose of 30 mg/kg provided a consistent decrease in both platelet accumulation and fibrin formation in this thrombosis assay over VPA, indicating that compound Ia is beneficial in treating or preventing conditions associated with excess fibrin deposition when compared with VPA and Compound Ib. In addition, compound Ia and compound Ib administered at a dose of 100 mg/kg provided consistent decrease in platelet accumulation over VPA in this thrombosis assay (FIG. 4).

### Example 4—Formation of 2-Propyl-4-pentenoic acid (4-ene metabolite)

Formation of 2-Propyl-4-pentenoic acid (4-ene metabolite) in Cyp 2C9 in vitro experiments with VPA and compounds of the invention. VPA, compound Ia and compound Ib (all compounds at 1 mM) were incubated with Human CYP2C9 0.2 pmol/μl bactosomes (Cypex, UK) in 100 mM potassium phosphate buffer pH 7.4 containing 5 mM magnesium chloride and 2 mM NADPH. Samples were withdrawn before addition of NADPH (time 0) and at different time points (30, 60 and 180 minutes) after addition of NADPH. Samples were added to the same volume ice cold acetonitrile, vortexed, centrifuged at 10000×g and stored at +4° C. until analysis. To increase the analytical sensitivity, the sample supernatants were derivatized with N-(3 Dimethylaminopropyl)-N′-ethylcarbodiimide) and 3-Nitrophenylhydrazibe hydrochloride. Resulting products were analysed using LC-MS/MS (UHPLC Agilent 6495, XSelect HSS T3 XP Column). The area under the curve calculations of formed 4-ene metabolite were based on the trapetzoidal rule, and the formation expressed as % 4-ene*min. The amounts of formed 4-ene metabolite for compounds Ia and Ib were compared with the formed amount 4-ene metabolite for VPA. For compound Ia, the reduction of formed 4-ene metabolite was 96.7% when compared with VPA. For compound Ib, the reduction of formed 4-ene metabolite was 88.8% when compared with VPA (see FIG. 5).

1. A compound of Formula I 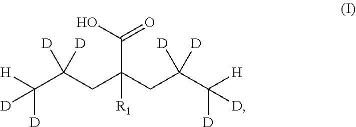
wherein R₁ is either H or D and wherein D is deuterium,
or a pharmaceutically acceptable salt thereof.

2. The compound of claim 1, wherein R₁ is H.

3. The compound of claim 1, wherein R₁ is D.

4. The compound of claim 1, wherein the pharmaceutically acceptable salt is a sodium salt.

5. A pharmaceutical composition comprising the compound of claim 1 and at least one pharmaceutical vehicle.

6. A method for the treatment of an abnormal condition associated with thrombus formation, excess fibrin deposition and/or fibrosis in a subject in need thereof comprising administering a compound of claim 1 or a pharmaceutically acceptable salt thereof.

7. A method for the treatment of bipolar disorder comprising administering a compound of claim 1 or a pharmaceutically acceptable salt thereof.

8. A method for the treatment of migraines comprising administering a compound according to claim 1 or a pharmaceutically acceptable salt thereof.

9. A method for treating epilepsy comprising administering a compound according to claim 1 or a pharmaceutically acceptable salt thereof.

10. A method of treating an abnormal condition associated with thrombus formation, excess fibrin deposition and/or fibrosis in a subject in need of treatment thereof comprising administering a therapeutically effective amount of the pharmaceutical composition of claim 5 to the subject.

11. The method of claim 10, wherein the abnormal condition associated with thrombus formation and/or excess fibrin deposition is selected from the group consisting of atherosclerosis, myocardial infarction, ischemic stroke, deep vein thrombosis, superficial vein thrombosis, thrombophlebitis, pulmonary embolism, disseminated intravascular coagulation, renal vascular disease and intermittent claudication, and wherein the abnormal condition associated with fibrosis is selected from the group consisting of cardiac fibrosis, arterial fibrosis, pulmonary fibrosis, fibrosis associated with pulmonary arterial hypertension, fibrosis associated with thromboembolic disease, fibrosis associated with NASH, kidney fibrosis, eye fibrosis, skin fibrosis, liver fibrosis, pancreas fibrosis and other GI tract fibrosis.

12. The method of claim 10, wherein the compound for use or pharmaceutical composition is administered to the subject through a route of administration selected from the group consisting of oral, intravenous, intraperitoneal, intradermal, intramuscular, subcutaneous, intranasal, epidural, oral, sublingual, intranasal, intracerebral, transdermal, rectal and topical.

13. (canceled)

14. (canceled)

15. (canceled)

16. A method of treating bipolar disorder in a subject in need of treatment thereof comprising administering a therapeutically effective amount the pharmaceutical composition of claim 5 to the subject.

17. The method of claim 16, wherein the compound for use or the pharmaceutical composition is administered to the subject through a route of administration selected from the group consisting of oral, intravenous, intraperitoneal, intradermal, intramuscular, subcutaneous, intranasal, epidural, oral, sublingual, intranasal, intracerebral, transdermal, rectal and topical.

18. A method of reducing the frequency of migraine headaches in a subject in need of reduction thereof comprising administering a therapeutically effective amount of the pharmaceutical composition of claim 5 to the subject.

19. The method of claim 18, wherein the compound for use or the pharmaceutical composition is administered to the subject through a route of administration selected from the group consisting of oral, intravenous, intraperitoneal, intradermal, intramuscular, subcutaneous, intranasal, epidural, oral, sublingual, intranasal, intracerebral, transdermal, rectal and topical.

20. A method of treating epilepsy in a subject in need of treatment thereof comprising administering a therapeutically effective amount the pharmaceutical composition of claim 5 to the subject.

21. The method of claim 20, wherein the pharmaceutical composition is administered to the subject through a route of administration selected from the group consisting of oral, intravenous, intraperitoneal, intradermal, intramuscular, subcutaneous, intranasal, epidural, oral, sublingual, intranasal, intracerebral, transdermal, rectal and topical.
